(19) United States
(12) Patent Application Publication
(10) Pub. No.: US 2022/0177925 A1
(43) Pub. Date: Jun. 9, 2022

(54) Visualized Screening Method for Aspergillus Recombinant Strains with Multigene Editing

(71) Applicant: Jiangnan University, Wuxi (CN)

(72) Inventors: Song Liu, Wuxi (CN); Cen Li, Wuxi (CN); Guocheng Du, Wuxi (CN); Jian Chen, Wuxi (CN); Jingwen Zhou, Wuxi (CN)

(21) Appl. No.: 17/676,278

(22) Filed: Feb. 21, 2022

Related U.S. Application Data
(63) Continuation of application No. PCT/CN2020/110404, filed on Aug. 21, 2020.

(30) Foreign Application Priority Data
Aug. 21, 2019 (CN) 2019107731937

(51) Int. Cl. C12N 15/90; C12N 9/22; C12N 15/11; C12N 15/80; C12N 1/14

(52) U.S. Cl. CPC C12N 15/902; C12N 9/22; C12N 15/11; C12N 2800/10; C12N 1/14; C12N 2310/20; C12N 2800/80; C12N 15/80

(57) ABSTRACT

The present disclosure discloses a visualized screening method for an Aspergillus recombinant strain with multigene editing and belongs to the technical field of gene engineering. CRISPR-Cas9 is used in the disclosure to cleave spore color change-related genes and a target gene in Aspergillus at the same time, such that editing of the target gene is visualized and an Aspergillus niger strain with multigene editing can be rapidly and efficiently screened out through spore phenotypes. Through different combinations of visualized genes and non-phenotypic change genes, rapid screening of the strain with multigene editing and simultaneous screening of multiple visualized genes are realized, and use of resistance genes in industrial strains is reduced.

### TECHNICAL FIELD

The present disclosure relates to a visualized screening method for Aspergillus recombinant strains with multigene editing and belongs to the technical field of genetic engineering.

### BACKGROUND

Filamentous fungi have a long history of development in the traditional fermentation industry. Aspergillus is used for food fermentation and production of enzymes and organic acids. Commercially valuable Aspergillus species include Aspergillus niger, Aspergillus oryzae, etc. However, a molecular modification process of industrial Aspergillus strain is hindered by lack of an efficient gene editing method and a method for rapidly screening recombinant strains.

Aspergillus cells are wrapped by hyphae, have a cell wall structure more complex compared with prokaryotic cells such as Escherichia coli and Bacillus subtilis. Therefore, visualized screening of single Aspergillus colony is difficult to realize. In the common visualized screening method, green fluorescent protein (GFP) was fused to N-terminal target sequence, and screening by fluorescence. In prokaryotic cells such as Escherichia coli and Bacillus subtilis, single recombinant colony can be picked by green fluorescence deposition of the microorganisms, or single cell is sorted by flow cytometer. However, when the GFP was expressed in Aspergillus for sorting, a fluorescence microscope is needed to observe fluorescence of the microorganisms, which is difficult to realize sorting. Screening Aspergillus recombinant strain by color reaction of chromogenic culture medium not only has use limitation, but also has complex procedure and inaccurate effect. Therefore, a more rational “visualized” procedure is needed for screening Aspergillus recombinant strains.

Different types of Aspergillus have different color of spores. The different colors of the spores can be used as visualized features to distinguish Aspergillus. The color of host spores can be changed by editing spore pigmentation related genes, which are used as a “visualized” screening marker for Aspergillus recombinant strain. Generally, mature spores of Aspergillus niger wild type strains are black. When the genes related to spore color change and a target gene are simultaneously mutated in Aspergillus using CRISPR-Cas9, the target gene editing can be visualized.

### SUMMARY

A first object of the present disclosure is to disclose a visualized screening method for Aspergillus strains with gene editing, in which a CRISPR-Cas9 gene editing technology is used to simultaneously knock out genes (a) and (b) in the Aspergillus, where (a) are genes affecting a spore color change and (b) are genes with unchanged phenotypes of Aspergillus before and after knockout; and

the genes affecting a spore color change are fwnA, pptA and/or brnA.

Preferably, the gene affecting a spore color change is fwnA.

In one implementation, the genes with unchanged phenotypes of Aspergillus before and after knockout include, but are not limited to, amyA, ammA, pepA and kusA.

Preferably, the genes with unchanged phenotypes of Aspergillus before and after knockout are amyA and/or ammA.

In one embodiment, the fwnA has a nucleotide sequence as set forth in SEQ ID NO:24.

In one embodiment, the pptA has a Gene ID of 4985743 (a nucleotide sequence as set forth in SEQ ID NO:50) and the brnA has a Gene ID of 4987395 (a nucleotide sequence as set forth in SEQ ID NO:51).

In one embodiment, the amyA has a Gene ID of 4980947 (a nucleotide sequence as set forth in SEQ ID NO:52), the ammA has a Gene ID of 4984565 (a nucleotide sequence as set forth in SEQ ID NO:53), the pepA has a Gene ID of 4987328 (a nucleotide sequence as set forth in SEQ ID NO:54), and the kusA has a Gene ID of 4987871 (a nucleotide sequence as set forth in SEQ ID NO:55).

In one embodiment, Aspergillus includes Aspergillus flavus, Aspergillus niger, Aspergillus fumigatus, Aspergillus versicolor and Aspergillus nidulans.

Preferably, the host is Aspergillus niger; more preferably, the host is Aspergillus niger CCTCC M 2018881, which has been disclosed in Chinese Patent Application No. CN110438018A.

In one embodiment, the method is to construct a Cas9 expression plasmid and an sgRNA expression cassette and transfer the Cas9 expression plasmid and the sgRNA expression cassette into Aspergillus niger for co-expression.

In one embodiment, a codon-optimized Cas9 gene sequence of Aspergillus niger is as set forth in SEQ ID NO:4.

In one embodiment, the method is to construct an sgRNA expression cassette by using a Pu3 or Pu6 promoter and a corresponding Tu3 or Tu6 terminator.

In one embodiment, the Pu6 promoter has a nucleotide sequence as set forth in SEQ ID NO:3; and the Tu6 terminator has a nucleotide sequence as set forth in SEQ ID NO:5.

In one embodiment, the Pu3 promoter has a nucleotide sequence as set forth in SEQ ID NO:2; and the Tu3 terminator has a nucleotide sequence as set forth in SEQ ID NO:4.

In one embodiment, sgRNA is released by using Aspergillus endogenous tRNAs.

In one embodiment, the endogenous tRNAs include tRNA^{Ala}, tRNA^{Arg}, tRNA^{Cys}, tRNA^{Ile}, tRNA^{Leu}, tRNA^{Lys}, tRNA^{Met}, tRNA^{Phe}, tRNA^{Ser}, tRNA^{Thr}, tRNA^{Val}, tRNA^{Glu}, tRNA^{Pro}, tRNA^{Glu}, tRNA^{Gln} and tRNA^{Gly}.

In one embodiment, when a single gene is edited, a first tRNA after a promoter is preferably tRNA^{Ala}.

In one embodiment, when two genes are edited, a first tRNA after a promoter is preferably tRNA^{Ala} and a second tRNA is preferably tRNA^{Arg} or tRNA^{Phe}.

In one embodiment, when multiple genes are edited, a first tRNA after a promoter is preferably tRNA^{Ala}, a second tRNA is preferably tRNA^{Phe}, and a tRNA in a last sgRNA is preferably tRNA^{Arg} or tRNA^{Ile}.

In one embodiment, the tRNA^{Ala} has a nucleotide sequence as set forth in SEQ ID NO:10; the tRNA^{Arg} and the tRNA^{Phe} respectively have a nucleotide sequence as set forth in SEQ ID NO:20 and SEQ ID NO:16; and the tRNA^{Ile} has a nucleotide sequence as set forth in SEQ ID NO:19.

A second object of the present disclosure is to disclose a visualized system for gene knockout in Aspergillus, the system includes a gene encoding a Cas9 protein, an sgRNA expression cassette and a screening marker; the sgRNA expression cassette contains target sequences of genes affecting a spore color change and target sequences of genes not affecting phenotypes of Aspergillus.

In one embodiment, sgRNA is released by using Aspergillus endogenous tRNAs.

In one embodiment, the endogenous tRNAs include tRNA^{Ala}, tRNA^{Arg}, tRNA^{Cys}, tRNA^{Ile}, tRNA^{Leu}, tRNA^{Lys}, tRNA^{Met}, tRNA^{Phe}, tRNA^{Ser}, tRNA^{Thr}, tRNA^{Val}, tRNA^{Glu}, tRNA^{Pro}, tRNA^{Glu}, tRNA^{Gln} and tRNA^{Gly}.

In one embodiment,

when a single gene is edited, a first tRNA after a promoter is tRNA^{Ala};

when two genes are edited, a first tRNA after a promoter is tRNA^{Ala} and a second tRNA is tRNA^{Arg};

when multiple genes are edited, a first tRNA after a promoter is tRNA^{Ala}, a tRNA in a last sgRNA is tRNA^{Arg} or tRNA^{Ile}; or a first tRNA after a promoter is tRNA^{Ala}, a tRNA in a last sgRNA is tRNA^{Arg} or tRNA^{Ile} and a Pu6 promoter and a Tu6 terminator are used to separately promote and terminate an expression of the last sgRNA;

the Pu6 promoter has a nucleotide sequence as set forth in SEQ ID NO:3; the Tu6 terminator has a nucleotide sequence as set forth in SEQ ID NO:5; and

the tRNA^{Ala} has a nucleotide sequence as set forth in SEQ ID NO:10; the tRNA^{Arg} and the tRNA^{Phe} respectively have a nucleotide sequence as set forth in SEQ ID NO:20 and SEQ ID NO:16; and the tRNA^{Ile} has a nucleotide sequence as set forth in SEQ ID NO:19.

A third object of the present disclosure is to disclose a method for improving screen efficiency of gene editing in Aspergillus, in which a CRISPR-Cas9 gene editing technology is used to simultaneously knock out genes (a) and (b) in the Aspergillus, where (a) are genes affecting a spore color change; (b) are genes with unchanged phenotypes of Aspergillus before and after knockout; the genes affecting a spore color change include fwnA, pptA and brnA; and the genes with unchanged phenotypes of Aspergillus before and after knockout include, but are not limited to, amyA, ammA, pepA and kusA.

The present disclosure also discloses use of a visualized screening method for an Aspergillus strain with gene editing in gene editing of Aspergillus.

The present disclosure also discloses use of a gene knockout visualized system for Aspergillus in gene editing of Aspergillus.

The present disclosure also discloses use of a method for improving screen efficiency in gene editing of Aspergillus in gene editing of Aspergillus.

In the present disclosure, CRISPR-Cas9 is used to cleave spore color change-related genes and a target gene in Aspergillus at the same time, such that editing of the target gene is visualized and an Aspergillus niger strain with multigene editing can be rapidly and efficiently screened out through spore phenotypes. Through different combinations of visualized genes and non-phenotypic change genes, rapid screening of the strain with multigene editing and simultaneous screening of multiple visualized genes are realized, and use of resistance genes in industrial strains is reduced. The method of the present disclosure can also be generally used in other Aspergillus species and a target gene can be quickly and accurately knocked out by combining visualized genes with a color change and the target gene to be knocked out.

### BRIEF DESCRIPTION OF FIGURES

FIG. 1 is a map of an Aspergillus niger Cas9 expression plasmid pUC19-Cas9.

FIG. 2 is a map of an Aspergillus niger sgRNA co-expression plasmid pUC19-sgRNA.

FIG. 3A is a map of an Aspergillus niger non-phenotypic gene sgRNA and a visualized phenotypic gene sgRNA co-expression plasmid pUC19-sgRNA-1.

FIG. 3B is a map of an Aspergillus niger non-phenotypic gene sgRNA and a visualized phenotypic gene sgRNA co-expression plasmid pUC19-sgRNA-2.

FIG. 3C is a map of an Aspergillus niger non-phenotypic gene sgRNA and a visualized phenotypic gene sgRNA co-expression plasmid pUC19-sgRNA-3.

FIG. 4 shows comparison of a spore color of an Aspergillus niger visualized transformant with gene editing.

### DETAILED DESCRIPTION

(I) Culture Medium

PDA culture medium: 200 g of potatoes, 20 g of glucose, 15-20 g of agar and the balanced water to a constant volume of 1 L.

LB culture medium: 10 g of peptone, 5 g of yeast powder, 10 g of NaCl and the balanced water to a constant volume of 1 L.

(II) Reagent Formula

STC buffer solution: 1.2 M sorbitol, 50 mM CaCl₂ and 10 mM Tris, and pH of 7.5-8.

PEG buffer solution: 25% PEG 6000, 50 mM CaCl₂ and 10 mM Tris, and pH of 7.5-8.

TABLE 1
PCR primers for verification of targeted sites

| Name of | | |
|---|---|---|
| primers | Sequence | |
| pptA-F | TAACCCAACCCCTCACTTCACCT | SEQ ID NO: 25 |
| pptA-R | TGGAGACGTATTCCAGGAAGGCT | SEQ ID NO: 26 |
| brnA-F | TGTTTGGATCTGATGCCGAGGC | SEQ ID NO: 27 |
| brnA-R | GGCTTGACGCTGATCTTGGT | SEQ ID NO: 28 |
| fwnA-F | GACCAATGACAAGACTCTGTGGGT | SEQ ID NO: 29 |
| fwnA-R | TCTTCTTCCCCTCCGCAGTGAC | SEQ ID NO: 30 |
| kusA-F | TCAAATGCGCCTATCACTTCATGC | SEQ ID NO: 31 |
| kusA-R | CCGCCGGTTAATACGATGTCATAT | SEQ ID NO: 32 |
| amyA-F | GCAGGGCATCATCGACAAGGT | SEQ ID NO: 33 |
| amyA-R | GGTGGTATCGAGATCAGGCAAGG | SEQ ID NO: 34 |
| pepA-F | TCCATCATGACGGCTGCCA | SEQ ID NO: 35 |
| pepA-R | CGAACTCGGAGCTGATCTTGC | SEQ ID NO: 36 |
| ammA-F | GACGCTGTTCTGTCGCTTTGT | SEQ ID NO: 37 |
| ammA-R | GATCAGGCAGTATGGGTGGAAGT | SEQ ID NO: 38 |

TABLE 2
Sequence of tRNA

| Name | Sequence | |
|---|---|---|
| tRNA^{Pro} | GCCCGGGTGGTCTAGTGGTATGATTCTCGCTTAGGGATAC | SEQ ID NO: 9 |
| | AAACCCAAGCATATCTGCGAGAGGTCCCGCGTTCGATCCG | |
| | CGGCTCGGGCC | |
| tRNA^{Ala} | GGGGCTGTGGTTTAGTGGTATAATATTCCCTTAGCATGGG | SEQ ID NO: 10 |
| | AGAGGtCCGGGGTTCGATTCCCCGCAGCTCCA | |
| tRNA^{Gly} | ACAACCATACGTTAATTGGTAAACTAGTCGTCTTCCAAAC | SEQ ID NO: 11 |
| | GATAAtTGTGAGTTCGAACCTCACTGGTTGTA | |
| tRNA^{Thr} | GCTTCTATGGCTCAGTTGGTAGAGCGCATGACTAGTAATC | SEQ ID NO: 12 |
| | ATGAGGtCCGCGGTTCGAATCCGCGTGGAAGCA | |
| tRNA^{Val} | GGCCGGATGGTGTAGTTGGTtATCACGTATCGTTAACACC | SEQ ID NO: 13 |
| | GATAAGGtCCTGGGATCGAGCCCCAGTCTGGTCA | |
| tRNA^{Ser} | GTCAGTGTGGCCGAGTGGTtAAGGCGATAGACTAGAAATCT | SEQ ID NO: 14 |
| | ATTGGGTTCGCCCGCACAGGTTCGAGTCCTGTCGCTGACG | |
| tRNA^{Leu} | GGCAAGATGGCCGAGTTGGTCCAAGGCGTCAGGTTAAGGT | SEQ ID NO: 15 |
| | CCACCTTAATACCCAGCTTCACAGCTTCCTGATCATCGTA | |
| | AGATGGCGTGGGTTCGAATCCCACTCTTGTCA | |
| tRNA^{Phe} | GGGGCAATGGCGCATCTGGGAGCGCGTCAGACTGAAGATC | SEQ ID NO: 16 |
| | TGGAGGtGGCCGGTTCAAGCCCGGCTTGCCCCA | |
| tRNA^{Lys} | GCCCGGCTAGCTCAATCGGTAGAGCGTGAGACTCTTAATC | SEQ ID NO: 17 |
| | TCAAGGcTGCGGGTTCGAGCCCCGCGTTGGGCT | |
| tRNA^{Glu} | TCCGATATGGTGTAGTGGCtAACATCGCCGTCTCTCACAC | SEQ ID NO: 18 |
| | GGCAGCCGGGGGTTCGATTCCCCCTATCGGAG | |
| tRNA^{Ile} | GGTCCCATAGCTCAGTTGGTtAGAGCGTGACGCTAATAAC | SEQ ID NO: 19 |
| | GTCAAAGtCGAGGGTTCGAGCCCCTCTGGGACCA | |
| tRNA^{Arg} | GGCCTGCTGGCCCAATGGTAAGGCGCTTGACTACGGATCA | SEQ ID NO: 20 |
| | AGAGAtTGCAGGTTCGAGTCCTGCGTAGGTCA | |
| tRNA^{Met} | AGCATGTTAGCTCAGGGGAAGAGCGCCGGGCTCATAACCC | SEQ ID NO: 21 |
| | GGAGGtCCCTGGATCGAAACCAGGACATGCTA | |
| tRNA^{Gln} | GGTTGTGTAGTGTAATGGTcATCACTCTGGATTCTGATTC | SEQ ID NO: 22 |
| | CAGCAATCCCGGTTCGATCCCGGGCACGACCT | |
| tRNA^{Cys} | GGGCCGGTAGCTCAGGGGTAGAGCGTGGGACTGCAGATCT | SEQ ID NO: 23 |
| | TAAGGtCACGCGTTCAAATCGCGTTCGGCCCT | |

### Example 1: Construction of Visualized Recombinant Aspergillus niger Strain with Single Gene Editing by Using CRISPR-Cas9

A CRISPR-Cas9 system included a gene encoding a Cas9 protein (a Cas9 gene), sgRNA and a screening marker.

(1) Construction of Cas9 Expression Vector

An Aspergillus promoter PglaA (with a nucleotide sequence as set forth in SEQ ID NO:7) or Ptef1 (with a nucleotide sequence as set forth in SEQ ID NO:6) and other Aspergillus strong promoters were used to express a Cas9 protein (with a nucleotide sequence as set forth in SEQ ID NO:1).

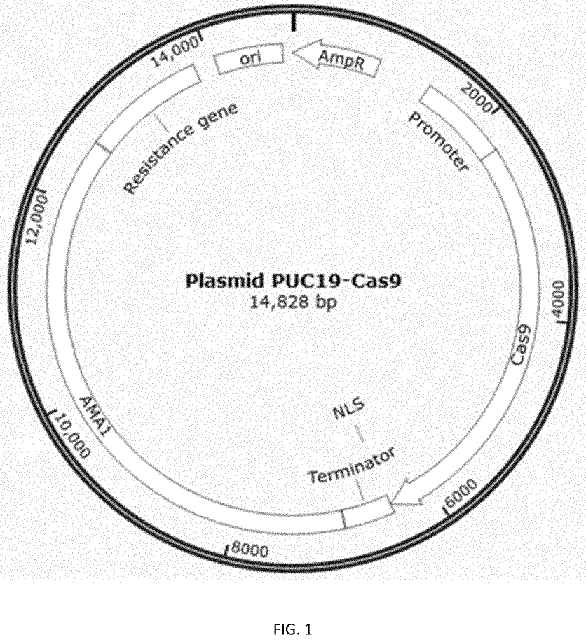

A ClonExpress® II One Step Cloning Kit (Vazyme) was used, pUC19 was used as a vector framework, an Aspergillus promoter sequence, a gene sequence encoding a Cas9 protein, a resistance gene and an AMA1 (GenBank: X78051.1) sequence were subjected to homologous recombination twice to obtain a Cas9 expression plasmid pUC19-Cas9 (a plasmid map was shown in FIG. 1). A nuclear localization signal (NLS) sequence (CCCAAGAAGAAGCGCAAGGTC, SEQ ID NO:56) was added at a or a C-terminal of a gene encoding a Cas9 protein (as set forth in SEQ ID NO:1).

(2) Construction of sgRNA Expression Cassette

A promoter Pu3, a protospacers sequence of a target gene (See Table 3), a gRNA backbone sequence (a nucleotide sequence as set forth in SEQ ID NO:8) and a terminator Tu3 are used to construct an sgRNA expression cassette.

TABLE 3
Sequence listing of target gene protospacers

| Target | | |
|---|---|---|
| gene | Target gene sequence | |
| fwnA | AGTGGGATCTCAAGAACTAC | SEQ ID NO: 39 |
| pptA | GGCGGGTGTCGATGTACCAC | SEQ ID NO: 40 |
| brnA | ACCATGCCAATGGATTCCGG | SEQ ID NO: 41 |
| kusA | CGAGCACTGGTAGATGATGA | SEQ ID NO: 42 |

Screening markers included filamentous fungal markers of similar effects as hygromycin B (hygB), orotidine-5′-phosphate dehydrogenase, acetamidase and the like commonly used in Aspergillus. A hygromycin resistance gene in a recombinant plasmid was obtained from a plasmid PAN7-1, expression cassette primers Hyg-F/R were shown in Table 4, and hyg in other resistance replaceable expression cassettes were selected for construction.

TABLE 4
Listing of primers

| Name of | | |
|---|---|---|
| primers | Primer sequence | |
| Hyg-F | GAATTCCCTTGTATCTCTAC | SEQ ID NO: 43 |
| | ACACAG | |
| Hyg-R | TGAAGAACGAATACCGCGAC | SEQ ID NO: 44 |
| | ATCCAACCCATC | |

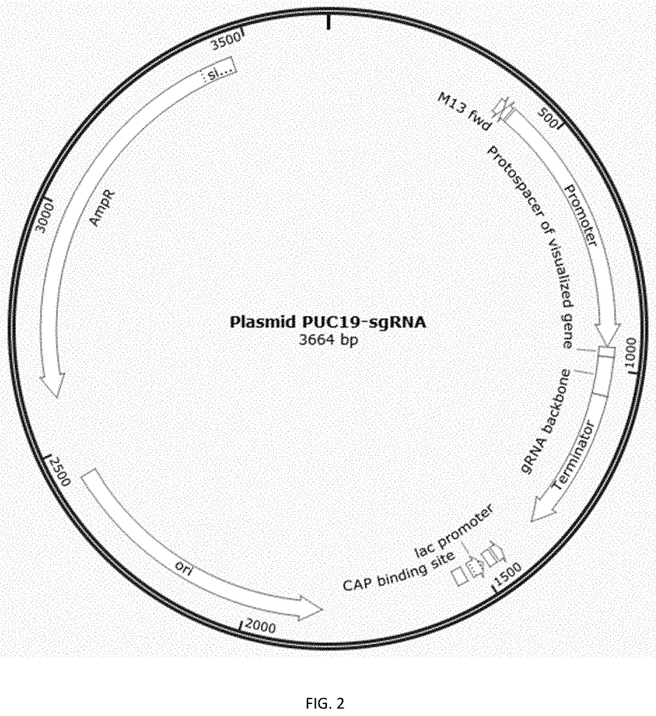

A ClonExpress® II One Step Cloning Kit (Vazyme) was used and pUC19 was used as a vector framework for recombination with the sgRNA expression cassette to construct an sgRNA expression plasmid (a plasmid map was shown in FIG. 2).

(3) Transformation of Cas9 Expression Plasmid and sgRNA Expression Cassette

The Cas9 expression plasmid and the sgRNA expression cassette were transferred into a host by using a protoplast transformation method.

Aspergillus niger hyphae were cultured in a PDA culture medium overnight and mycelia were collected and washed three times with normal saline; the washed mycelia were enzyme-digested with a Lysozyme for 3 h and filtered with four layers of lens paper to prepare a protoplast; the protoplast was collected by centrifugation at 4° C., and 1,000 rpm and washed 2-3 times with pre-cooled STC; and 100 μL of the prepared protoplast was taken, into which 10 μL of the Cas9 expression plasmid and 10 μL of the sgRNA expression cassette were added and mixed evenly, 2 mL of PEG 6000 was added, and the corresponding resistance was added to the culture medium for screening. The culture was conducted at 30° C. for 5-7 d, a genome of a transformant was extracted, and editing of a targeting site of a target gene was verified by PCR.

A positive single colony was picked and transferred to a plate, and each single colony was transferred three times (that is, a single colony was picked to a new culture medium for culture).

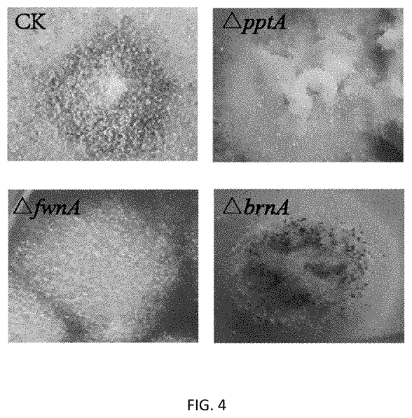

A spore color of a starting strain of Aspergillus niger CCTCC M 2018881 (the strain has been disclosed in the patent application document with the publication number of CN110438018A) was used as a control, changes in a color phenotype of Aspergillus niger spores after transformation were observed, after fwnA or pptA were destroyed, a spore color was changed from brown to white, and after brnA was destroyed, the spore color was changed from brown to olive (See FIG. 4). When a non-phenotypic change gene kusA was destroyed, phenotypes of microorganisms did not change. A spore color change transformant had significantly improved gene editing efficiency, where, a color mutant strain accounted for 14%, the color mutant strain has gene editing efficiency of brnA for 30% and editing efficiency of fwnA or pptA for 100% and editing efficiency of kusA for 4.16%.

### Example 2: Visualized Screening for Recombinant Aspergillus niger Strain with Double Gene Editing

(1) Construction of Cas9 Expression Vector

The specific implementation referred to step (1) in Example 1.

(2) Construction of sgRNA Expression Cassette

A Pu3 mutant promoter (to facilitate assembly of multiple sgRNAs, a BsaI site related with the sgRNAs was mutated to facilitate subsequent assembly, specifically a BsaI site mutation of a nucleotide sequence of the Pu3 promoter sequence as set forth in SEQ ID NO:2 was mutated from GAGACC to ACCCAC), target gene protospacers sequences pptA, fwnA and brnA (See Table 1), an amyA sequence (See Table 5), an sgRNA expression cassette realized by using tRNA^{Gly}, a gRNA backbone sequence (with a nucleotide sequence as set forth in SEQ ID NO:8), a terminator Tu3 (with a nucleotide sequence as set forth in SEQ ID NO:4) were used to construct the sgRNA expression cassette.

TABLE 5
Sequence listing of target gene protospacers

| Target | | |
|---|---|---|
| gene | Target gene sequence | |
| amyA | TCTCTTCGGCCCTTCATGAG | SEQ ID NO: 45 |

Screening markers included filamentous fungal markers of similar effects as hygromycin B (hygB), orotidine-5′-phosphate dehydrogenase, acetamidase and the like commonly used in Aspergillus. A hygromycin resistance gene in a recombinant plasmid was obtained from a plasmid PAN7-1, expression cassette primers Hyg-F/R were shown in Table 3, and hygB in other resistance replaceable expression cassettes were selected for construction.

A ClonExpress® II One Step Cloning Kit (Vazyme) was used, pUC19 was used as a vector framework, an Aspergillus promoter sequence, a gene sequence encoding a Cas9 protein, a resistance gene and an AMA1 sequence were subjected to homologous recombination twice to pUC19 to obtain a Cas9 expression plasmid pUC19-Cas9 (a plasmid map was shown in FIG. 1). A nuclear localization signal (NLS) sequence (CCCAAGAAGAAGCGCAAGGTC, SEQ ID NO:56) was added to a N-terminal or a C-terminal of a gene encoding a Cas9 protein (as set forth in SEQ ID NO:1).

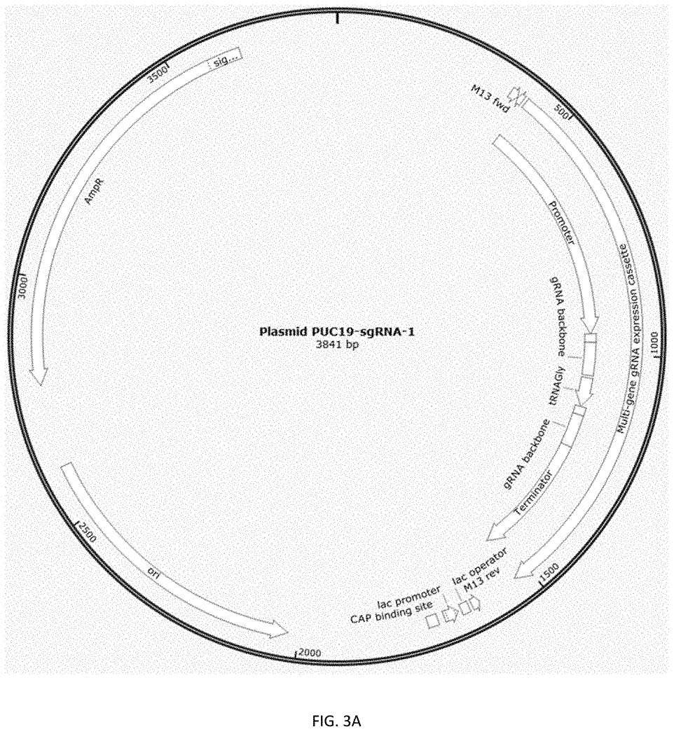

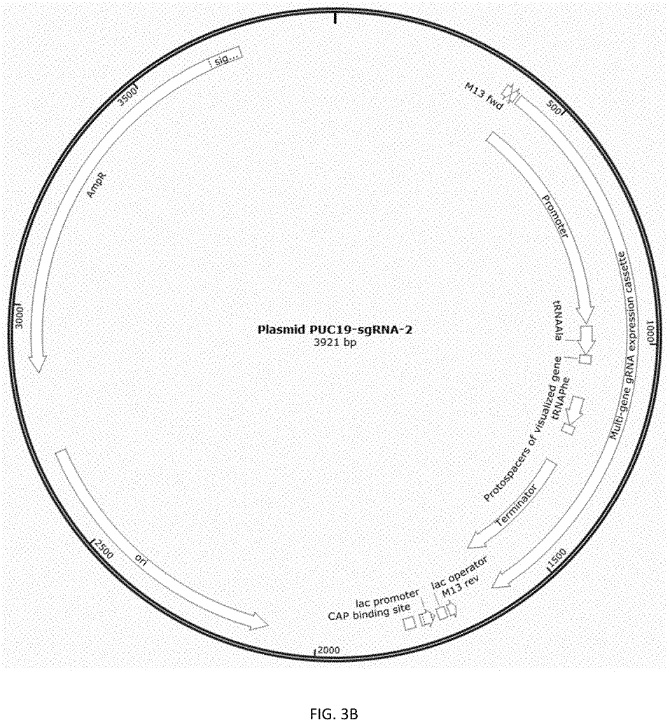

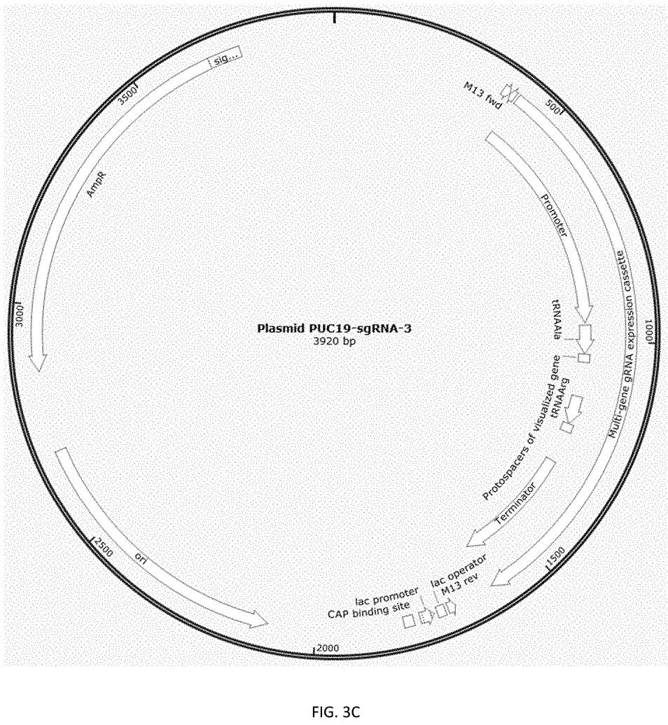

A ClonExpress® II One Step Cloning Kit (Vazyme) was used, pUC19 was used as a vector framework, an sgRNA expression cassette was recombined to the pUC19 to construct a double sgRNA expression plasmid PUC19-sgRNA-1 plasmid containing the sgRNA expression cassette with two protospacers sequences, and tRNA^{Gly} was used to release different sgRNAs. The sgRNA expression cassette included a visualized gene sgRNA and a non-phenotypic sgRNA, and the visualized gene sgRNA was pptA-sgRNA, fwnA-sgRNA or brnA-sgRNA, and the other non-phenotypic gene sgRNA was amyA-sgRNA (a plasmid map was shown in FIG. 3).

(3) Transformation of Cas9 Expression Plasmid and sgRNA Expression Cassette

The Cas9 expression plasmid and the double sgRNA expression cassette (including any one of pptA-sgRNA and amyA-sgRNA or fwnA-sgRNA and amyA-sgRNA, or brnA-sgRNA and amyA-sgRNA) were transferred into a host by protoplast transformation, and the sgRNA was directly connected with the tRNA to construct a strain containing the double sgRNA expression cassette.

Aspergillus niger hyphae were cultured in a PDA culture medium overnight and mycelia were collected and washed three times with normal saline; the washed mycelia were enzyme-digested with a Lysozyme for 3 h and filtered with four layers of lens paper to prepare a protoplast; the protoplast was collected by centrifugation at 4° C., and 1,000 rpm and washed 2-3 times with pre-cooled STC; and 100 μL of the prepared protoplast was taken, into which 10 μL of the Cas9 expression plasmid and 10 μL of the sgRNA expression cassette were added and mixed evenly, 2 mL of PEG 6000 was added, and the corresponding resistance was added to the culture medium for screening. The culture was conducted at 30° C. for 5-7 d and a single colony transformant of white spores were picked for sequencing verification.

A spore color of a starting strain of Aspergillus niger CCTCC M 2018881 was used as a control and changes of color phenotypes of Aspergillus niger spores after transformation were observed. After fwnA was knocked out, the spore color changed from black to white, a total of 4% of single colony on a plate showed white, the white single colony was directly picked, and the results showed that in the white single colony, a homozygous transformant with double-copy destroy of an amyA gene accounted for 25% and a positive selection rate of a transformant with multigene editing was improved. Editing of the double copies of the amyA gene did not require new resistance markers, which was beneficial to industrial production.

### Comparative Example 1

A same method was used as Example 2, a single-copy recombinant strain with gene amyA editing was constructed. A same method was used, the recombinant strain was cultured until a single colony was grown, the single colony was picked and gene editing efficiency was calculated. The gene editing efficiency was 5%.

### Example 3: Visualized Screening for Recombinant Aspergillus niger Strain with Multigene Editing

The specific implementation referred to Example 2. The difference was that tRNA^{Gly} behind a promoter in Example 2 was replaced by tRNA^{Ala} and a second tRNA was replaced by tRNA^{Phe}.

The tRNA^{Ala} and the tRNA^{Phe} were used to ligate with different sgRNAs. A visualized gene sgRNA expression cassette was a fwnA-sgRNA expression cassette and the other non-phenotypic gene sgRNA expression cassette was an amyA-sgRNA expression cassette (a plasmid map was shown in FIG. 3).

A spore color of a starting strain of Aspergillus niger CCTCC M 2018881 was used as a control and changes in color phenotypes of Aspergillus niger spores after transformation were observed. After fwnA was knocked out, the spore color changed from black to white, 36% of transformants had a white phenotype, the white single colony was directly picked, a positive selection rate of a transformant with multigene editing was improved, time for strain purification was saved, and the amyA gene editing efficiency was 90%. Editing of the amyA gene did not require new resistance markers, which was beneficial to industrial production.

### Comparative Example 2

A same method was used as Example 3, a recombinant strain with double gene fwnA and amyA editing was constructed by using tRNA^{Gly}. A same method was used, the recombinant strain was cultured until a single colony was grown, the single colony was picked and gene editing efficiency was calculated. The results showed that 6% was a white mutant and the amyA gene editing efficiency was 36%.

### Comparative Example 3

A same method was used as Example 3, a recombinant strain with fwnA and amyA gene editing was constructed by using tRNA^{Ala}. A same method was used, the recombinant strain was cultured until a single colony was grown, the single colony was picked and gene editing efficiency was calculated. The results showed that 37% was a white mutant and the amyA gene editing efficiency was 70%.

### Comparative Example 4

A same method was used as Example 3, a single copy recombinant strain with gene fwnA and amyA editing was constructed by using tRNA^{Phe}. A same method was used, the recombinant strain was cultured until a single colony was grown, the single colony was picked and gene editing efficiency was calculated. The results showed that 15% was a white mutant and the amyA gene editing efficiency was 80%.

### Example 4: Visualized Multi-Phenotype Screening for Recombinant Aspergillus Niger Strain with Gene Editing

The specific implementation referred to Example 2. The difference was that the amyA gene in Example 2 was replaced by pepA or ammA (sequence listing of protospacers was shown in Table 6), the tRNA^{Gly} behind a promoter in Example 2 was replaced by tRNA^{Ala} and a second tRNA was replaced by tRNA^{Arg}

TABLE 6
Sequence listing of target gene protospacers

| Target | | |
|---|---|---|
| gene | Target gene sequence | |
| pepA | CGGTGTCAAAGTCCAGATGG | SEQ ID NO: 46 |
| ammA | CTGCCCCAGGATACTGCTGA | SEQ ID NO: 47 |

The tRNA^{Ala} and the tRNA^{Arg} were used to release different sgRNAs. A visualized gene sgRNA expression cassette was a fwnA-sgRNA expression cassette and the other non-phenotypic sgRNA expression cassette was a pepA-sgRNA expression cassette or an ammA-sgRNA expression cassette (a plasmid map was shown in FIG. 3B).

The Cas9 expression plasmid and the sgRNA expression cassette were transferred into a host and obtained by sequencing.

A spore color of a starting strain of Aspergillus niger CCTCC M 2018881 was used as a control and changes in color phenotypes of Aspergillus niger spores after transformation were observed. After fwnA was knocked out, the spore color changed from black to white, a white single colony was picked, the gene editing efficiency was calculated, and the pepA or ammA gene editing efficiency was 75% and 80% respectively. The method was beneficial to perform an in-vivo screening for an active protospacers sequence of a target gene of Aspergillus niger, and saved time and cost of molecular operation.

### Example 5: Visualized Multi-Phenotype Screening for Recombinant Aspergillus Niger Strain with Gene Editing

The specific implementation referred to Example 2. The difference was that the amyA gene in Example 2 was replaced by ammA, at the same time, a third amyA-sgRNA (sequence listing of protospacers was shown in Table 7) was ligated with tRNA^{Ala}, the tRNA^{Gly} behind a promoter in Example 2 was replaced by tRNA^{Ala} and a second tRNA was replaced by tRNA^{Phe}.

TABLE 7
Sequence listing of target gene protospacers

| Target | | |
|---|---|---|
| gene | Target gene sequence | |
| ammA | CTGCCCCAGGATACTGCTGA | SEQ ID NO: 48 |
| amyA | TCTCTTCGGCCCTTCATGAG | SEQ ID NO: 49 |

The tRNA^{Ala}, the tRNA^{Arg} and the tRNA^{Phe} were used to release different sgRNAs. A visualized gene sgRNA was fwnA-sgRNA and the other two non-phenotypic gene sgRNAs were pepA-sgRNA and ammA-sgRNA, respectively.

The Cas9 expression plasmid and the sgRNA expression cassette were transferred into a host and obtained by sequencing.

A spore color of a starting strain of Aspergillus niger CCTCC M 2018881 was used as a control and changes in color phenotypes of Aspergillus niger spores after transformation were observed. After fwnA was knocked out, the spore color changed from black to white. In an optimized three-gene knockout white transformant, the ammA gene editing efficiency was 50%, the amyA gene editing efficiency was 100% and the double gene co-editing efficiency was 50%.

The method was beneficial to perform an in-vivo screening of an active protospacers sequence of multiple target genes of Aspergillus niger, and saved time and cost of molecular operation.

### Example 6: Visualized Multi-Phenotype Screening for Recombinant Aspergillus Niger Strain with Gene Editing

The specific implementation referred to Example 2. The difference was that the amyA gene in Example 2 was replaced by ammA, the tRNA^{Gly} behind a promoter in Example 2 was replaced by tRNA^{Ala} and a second tRNA^{Gly} was replaced by tRNA^{Phe}; and at the same time, an sgRNA expression cassette was added, tRNA^{Ile} was used for release, and a Pu6 promoter (with a nucleotide sequence as set forth in SEQ ID NO:3) and a Tu6 terminator (with a nucleotide sequence as set forth in SEQ ID NO:5) were used to express tRNA^{Ile} and ammA-sgRNA.

The tRNA^{Ala}, the tRNA^{Arg} and the tRNA^{Phe} were used to release different sgRNAs. A visualized gene sgRNA expression cassette was fwnA-sgRNA and the other two non-phenotypic gene sgRNAs were ammA-sgRNA and amyA-sgRNA.

The Cas9 expression plasmid and the sgRNA expression cassette were transferred into a host and subjected to sequencing verification.

A spore color of a starting strain of Aspergillus niger CCTCC M 2018881 was used as a control and changes in color phenotypes of Aspergillus niger spores after transformation were observed. After fwnA was knocked out, the spore color changed from black to white. In an optimized three-gene knockout white transformant, the ammA gene editing efficiency was 69%, the amyA gene editing efficiency was 100% and the two gene co-editing efficiency was 69%. The method was beneficial to perform an in-vivo screening of an active protospacers sequence of multiple target genes of Aspergillus niger, and saved time and cost of molecular operation.

Although the disclosure has been disclosed as above in the preferred examples, it is not intended to limit the disclosure. Any person skilled in the art can make various changes and modifications without departing from the spirit and scope of the disclosure. Therefore, the protection scope of the disclosure should be as defined in the claims.

What is claimed is:

1. A method for visualized screening of an Aspergillus strain with gene editing, wherein using CRISPR-Cas9 gene editing technology to simultaneously knock out genes (a) and (b) in the Aspergillus;
wherein (a) are genes affecting a spore color change; (b) are genes with unchanged phenotypes of Aspergillus before and after knockout; and
the genes affecting a spore color change are fwnA, pptA and/or brnA.

2. The method according to claim 1, wherein the genes with unchanged phenotypes of Aspergillus before and after knockout are amyA and/or ammA; and the amyA has a nucleotide sequence as set forth in SEQ ID NO:52 and the ammA has a nucleotide sequence as set forth in SEQ ID NO:53.

3. The method according to claim 2, wherein the fwnA has a nucleotide sequence as set forth in SEQ ID NO:24, the pptA has a nucleotide sequence as set forth in SEQ ID NO:50 and the brnA has a nucleotide sequence as set forth in SEQ ID NO: 51.

4. The method according to claim 3, wherein sgRNA is released by using Aspergillus endogenous tRNAs and the endogenous tRNAs comprise tRNA^{Ala}, tRNA^{Arg}, tRNA^{Cys}, tRNA^{Ile}, tRNA^{Leu}, tRNA^{Lys}, tRNA^{Met}, tRNA^{Phe}, tRNA^{Ser}, tRNA^{Thr}, tRNA^{Val}, tRNA^{Glu}, tRNA^{Pro}, tRNA^{Glu}, tRNA^{Gln} and tRNA^{Gly}.

5. The method according to claim 4, wherein when a single gene is edited, a first tRNA after a promoter is tRNA^{Ala}; and the tRNA^{Ala} has a nucleotide sequence as set forth in SEQ ID NO:10.

6. The method according to claim 4, wherein when two genes are edited, a first tRNA after a promoter is tRNA^{Ala} and a second tRNA is tRNA^{Arg} or tRNA^{Phe}; and the tRNA^{Arg} and the tRNA^{Phe} respectively have a nucleotide sequence as set forth in SEQ ID NO:20 and SEQ ID NO:16.

7. The method according to claim 4, wherein when multiple genes are edited, a first tRNA after a promoter is tRNA^{Ala}, a second tRNA is tRNA^{Phe}, and a tRNA in a last sgRNA is tRNA^{Arg} or tRNA^{Ile}; and the tRNA^{Ile} has a nucleotide sequence as set forth in SEQ ID NO:19.

8. The method according to claim 7, wherein a Pu6 promoter and a Tu6 terminator are used to separately promote and terminate an expression of the last sgRNA; the Pu6 promoter has a nucleotide sequence as set forth in SEQ ID NO:3; and the Tu6 terminator has a nucleotide sequence as set forth in SEQ ID NO:5.

9. The method according to claim 3, wherein the Aspergillus comprises Aspergillus flavus, Aspergillus niger, Aspergillus fumigatus, Aspergillus versicolor and Aspergillus nidulans.

10. A visualized system for gene knockout in Aspergillus, wherein the visualized system comprises a gene encoding a Cas9 protein, an sgRNA expression cassette and a screening marker; the sgRNA expression cassette contains target sequences of genes affecting a spore color change and target sequences of genes not affecting phenotypes of Aspergillus; and the genes affecting a spore color change comprise fwnA, pptA or brnA.

11. The visualized system according to claim 10, wherein the genes with unchanged phenotypes of Aspergillus before and after knockout are amyA and/or ammA; and the amyA has a nucleotide sequence as set forth in SEQ ID NO:52 and the ammA has a nucleotide sequence as set forth in SEQ ID NO:53.

12. The visualized system according to claim 11, wherein sgRNA is released by using Aspergillus endogenous tRNAs and the endogenous tRNAs comprise tRNA^{Ala}, tRNA^{Arg}, tRNA^{Cys}, tRNA^{Ile}, tRNA^{Leu}, tRNA^{Lys}, tRNA^{Met}, tRNA^{Phe}, tRNA^{Ser}, tRNA^{Thr}, tRNA^{Val}, tRNA^{Glu}, tRNA^{Pro}, tRNA^{Glu}, tRNA^{Gln} and tRNA^{Gly}.

13. The visualized system according to claim 12, wherein
when a single gene is edited, a first tRNA after a promoter is tRNA^{Ala};
when two genes are edited, a first tRNA after a promoter is tRNA^{Ala} and a second tRNA is tRNA^{Arg},
when multiple genes are edited, a first tRNA after a promoter is tRNA^{Ala}, a tRNA in a last sgRNA is tRNA^{Arg} or tRNA^{Ile}; or a first tRNA after a promoter is tRNA^{Ala}, a tRNA in a last sgRNA is tRNA^{Arg} or tRNA^{Ile} and a Pu6 promoter and a Tu6 terminator are used to separately promote and terminate an expression of the last sgRNA;
the Pu6 promoter has a nucleotide sequence as set forth in SEQ ID NO:3; the Tu6 terminator has a nucleotide sequence as set forth in SEQ ID NO:5; and
the tRNA^{Ala} has a nucleotide sequence as set forth in SEQ ID NO:10; the tRNA^{Arg} and the tRNA^{Phe} respectively have a nucleotide sequence as set forth in SEQ ID NO:20 and SEQ ID NO:16;
and the tRNA^{Ile} has a nucleotide sequence as set forth in SEQ ID NO:19.


SEQUENCE LISTING (56 sequences)

SEQ ID NO: 1 - DNA, 4104 bp, Artificial Sequence
  Synthetic DNA
atggacaagaagtatagcatcgggctggacattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtataaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgaccatatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgac

SEQ ID NO: 2 - DNA, 468 bp, Artificial Sequence
  Synthetic DNA
gatcacatagatgctcggttgacaggaccacctatagcaagtactttgtaggaggttctcctaacgcttggcttcaaatccaatcaggatatcagtgagttactgtttgtcatcgcatcctcatgaagtcgctcaacagccactgagaagcaaatatttgggagaccatccctgatgttgaaattttacgctggagcccattcaccggtgagctcggggacagtctggccagtggagcggaaaatcttcaactaactctgattggtaccacaggtagccccacagaacaaaccgagcaaacatgaaaattttcgcttgaggttagcgcactcgctagcgcctgcccgcaaatataaaaggccccgaaatcccatgcattttggaaatcaagcgactctacgtatgtcatccccctccctacctttgggaaaacctctcaactgcaagtcagaacattttgctaacagc

SEQ ID NO: 3 - DNA, 508 bp, Artificial Sequence
  Synthetic DNA
tggttcacttctctttagaaatcaactgtgggttttgctttttgcttcattctctttgtcttctccatctttgatcaaatcctggactttctcaatccccagctaattcaatcatagtcagttttctatttttattatttctttttcttttgaaatgtgattaacaaccagtccgttatatatcttgtacccagattacgcccaactcgtgctcctcagccacaaagatactcaattgatagccaagatacatacataccacaaagtaaggactccatgcattgagtattactcatcgtattctagactactccaaaactcagcacatagacaaacaatacgaacctcgtctaggggtgattcagaggcggcaaagcggggttttcgcatttgatgttcctggcacttatgtaagcccacgcttcccgctcaactaaaccatcagccaatcagactgctcagatttatcttttgaagggtaaataaatcattgtaaagaagaacaagt

SEQ ID NO: 4 - DNA, 226 bp, Artificial Sequence
  Synthetic DNA
tgtcaatttttacacttgatgttggtgtaatcaggcgatcagagtttgctcaaattctctcttgttttagattctgaagaatataatttttacatgactagttcattttacttccaagtagactatttgtagattgagattgccgtcaactttattcatagaatcacattaaattgctcattgacctgcatgtctatgcacacattcacatctatcttctcagggt

SEQ ID NO: 5 - DNA, 134 bp, Artificial Sequence
  Synthetic DNA
tttttgagcatttatcagcttgatatagaggtaggaatgtatggaggtgcagaatggctattttgttattggagcgggttcgaaacggagggcaggagactttttctaaatacgtcacgtgatatagagctgct

SEQ ID NO: 6 - DNA, 886 bp, Artificial Sequence
  Synthetic DNA
cgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcacc

SEQ ID NO: 7 - DNA, 1000 bp, Artificial Sequence
  Synthetic DNA
cctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagca

SEQ ID NO: 8 - DNA, 76 bp, Artificial Sequence
  Synthetic DNA
gttttagagctagaaatagcaagttaaaataaggctagtccgttatcaacttgaaaaagtggcaccgagtcggtgc

SEQ ID NO: 9 - DNA, 91 bp, Artificial Sequence
  Synthetic DNA
gcccgggtggtctagtggtatgattctcgcttagggatacaaacccaagcatatctgcgagaggtcccgcgttcgatccgcggctcgggcc

SEQ ID NO: 10 - DNA, 72 bp, Artificial Sequence
  Synthetic DNA
ggggctgtggtttagtggtataatattcccttagcatgggagaggtccggggttcgattccccgcagctcca

SEQ ID NO: 11 - DNA, 72 bp, Artificial Sequence
  Synthetic DNA
acaaccatacgttaattggtaaactagtcgtcttccaaacgataattgtgagttcgaacctcactggttgta

SEQ ID NO: 12 - DNA, 73 bp, Artificial Sequence
  Synthetic DNA
gcttctatggctcagttggtagagcgcatgactagtaatcatgaggtccgcggttcgaatccgcgtggaagca

SEQ ID NO: 13 - DNA, 74 bp, Artificial Sequence
  Synthetic DNA
ggccggatggtgtagttggttatcacgtatcgttaacaccgataaggtcctgggatcgagccccagtctggtca

SEQ ID NO: 14 - DNA, 81 bp, Artificial Sequence
  Synthetic DNA
gtcagtgtggccgagtggttaaggcgatagactagaaatctattgggttcgcccgcacaggttcgagtcctgtcgctgacg

SEQ ID NO: 15 - DNA, 112 bp, Artificial Sequence
  Synthetic DNA
ggcaagatggccgagttggtccaaggcgtcaggttaaggtccaccttaatacccagcttcacagcttcctgatcatcgtaagatggcgtgggttcgaatcccactcttgtca

SEQ ID NO: 16 - DNA, 73 bp, Artificial Sequence
  Synthetic DNA
ggggcaatggcgcatctgggagcgcgtcagactgaagatctggaggtggccggttcaagcccggcttgcccca

SEQ ID NO: 17 - DNA, 73 bp, Artificial Sequence
  Synthetic DNA
gcccggctagctcaatcggtagagcgtgagactcttaatctcaaggctgcgggttcgagccccgcgttgggct

SEQ ID NO: 18 - DNA, 72 bp, Artificial Sequence
  Synthetic DNA
tccgatatggtgtagtggctaacatcgccgtctctcacacggcagccgggggttcgattccccctatcggag

SEQ ID NO: 19 - DNA, 74 bp, Artificial Sequence
  Synthetic DNA
ggtcccatagctcagttggttagagcgtgacgctaataacgtcaaagtcgagggttcgagcccctctgggacca

SEQ ID NO: 20 - DNA, 72 bp, Artificial Sequence
  Synthetic DNA
ggcctgctggcccaatggtaaggcgcttgactacggatcaagagattgcaggttcgagtcctgcgtaggtca

SEQ ID NO: 21 - DNA, 72 bp, Artificial Sequence
  Synthetic DNA
agcatgttagctcaggggaagagcgccgggctcataacccggaggtccctggatcgaaaccaggacatgcta

SEQ ID NO: 22 - DNA, 72 bp, Artificial Sequence
  Synthetic DNA
ggttgtgtagtgtaatggtcatcactctggattctgattccagcaatcccggttcgatcccgggcacgacct

SEQ ID NO: 23 - DNA, 72 bp, Artificial Sequence
  Synthetic DNA
gggccggtagctcaggggtagagcgtgggactgcagatcttaaggtcacgcgttcaaatcgcgttcggccct

SEQ ID NO: 24 - DNA, 6680 bp, Artificial Sequence
  Synthetic DNA
atggagggtccatctcgtgtgtacctttttggagaccagaccagcgacatcgaagctggcctgcgccgtctgctccaagcgaagaatagtaccattgtccagtcctttttccagcaatgcttccatgcaattcgtcaagagatcgcgaagctcccgccgtctcatcggaagctcttcccacgcttcacgagcatcgttgatctcctttccaggagtcgtgaatcaggtcctagccctgtcctggagagtgcattgacatgcatctaccaattgggttgtttcattcagtaagtcaatgagttaccatctatacttgacaagtctgaccagccttcagcttttacggggatcttggacatgactaccctacaccctccaacagccatcttgttggcctgtgcactggtgttctgagctgcacggctgtaagttgcgccagaaatgttggagagcttattccagctgcagtggaatcggttgtaattgcactgcgactgggaatctgcgtttttcgagttcgagaactggtggactccgccgattccgagtcaacatgctggtcagcgttggtttctggaatcagtgaagcagaggctagccacctgatcgacgagtacagtagtaagaaggtgtgctcttccaactttaaacccccgcattgtgggatgctgacagatgcaggctactccgccttcttcgaaaccgtatatcagcgcggtaagctctaatggcgttactgtcagcgcaccacctacggtacttgatgaattcgtcgagacctgcatttccaagaattacaagccagtgaaggcccctattcatggcccgtaccatgcgccacatctgtatgatgataaggatatcgaccgcatcctgcagcagtcctctgctctagaaggactgaccggctgttcacccgttattcccatcatctccagtaacactggaaagccgatcaaggccaagtccatcaaagatctcttcaaggtcgcactggaggagatactcctacgacgactatgctgggacaaggtcacggagtcctgcacatcagtctgcaagaccggcacaaaccactcttgcaaattgtttccgatctcgagtagcgccactcaaagtttgttcacagtcctcaagaaggccggtgtgagcatcagcttggagactggggtaggagagatcgcgacgaacccagaaatgcggaaccttactggcaaggcagaaaattcaaagattgctatcattggtatgtctggaagatttcctgactcggatggtacggagagcttctggaacctcctgtacaaaggactcgacgtacatcgcaaagtccccgcagaccgttgggacgttgatgcccacgtcgacatgaccgggtcaaagagaaacacaagcaaagtggcttacggttgctggatcaacgaacccggcctgtttgacccccgattcttcaacatgtcgcctcgggaagcactccaagcagatcctgcacaacgtcttgcgttgcttacagcgtacgaggctctcgagatggctggcttcatcccggatagctctccatcgacgcagagggaccgtgtgggtattttctacggaatgaccagtgacgactaccgtgagatcaacagcggccaggacattgatacctatttcatccctggcggtaaccgagcatttacgccgggtcggataaactactacttcaaatttagcggccccagtgtgagcgttgacacagcgtgctcgtctagtcttgctgctatccacatggcttgcaattcgatctggagaaatgactgcgatgccgccatcactggaggtgtgaacattctgaccagccctgacaaccacgccggtctggatcggggccatttcctgtccaccactggcaactgtaacacctttgatgacggcgccgacggctactgtagagcggacggagttggaagcatcgttttgaagcggcttgaagatgccgaggccgacaacgacccgatcctggccgtcatcaacggtgcttacaccaaccactcggcggaggccgtgtcaatcactcgtccccatgttggcgcgcaagcattcatcttcaacaagctgctcaatgatgcgaatatcgaccctaaggacgtgagctacgtggaaatgcatggcactggaactcaagcaggtgatgcagtcgaaatgcagtccgttcttgacgtcttcgcaccagactaccgccggggtcccggtcaatcgcttcatatcggttctgccaaggcaaacattggacacggtgaatccgcatcaggagtgactgctcttgtcaaggtcctcctaatgatgagagagaacatgattcctcctcattgtggtatcaagaccaagatcaattccaatttcccgacagacttggcgaagcgcaatgttcatatcgccttccaacccactccctggaatcggccagcttcaggaaagcggcgaactttcgtcaacaacttttctgctgctggtggtaacactgctcttctactggaagatgctcccataccggaacgccaagggcaggaccccaggtcgttccatttggtctccgtgtcagcaagatcccagtctgcattgaagaacaacgtcgaagctctggtgaagtacattgactctcagggcaagtcctttggtgtgaaagagactgaattccttccaaacctggcgtacacgaccaccgcacgccgtatccaccatcccttccgtgtcattgcggttggagcgaacctacaatcactgcgtgactcgctgcatggtgctttgcaccgtgagacatataccccagttccctcaacggctcctggtattggtttcgtcttcaccggccaaggagcccaatactccggaatgggcaaggaactctaccgcagttgtttccaattccgaaccaccattgagcattttgactgcatcgcaagaagccagggccttccttctatccttcctcttgtcgatggaagcgtggctgtcgaagaacttagccctgtcgtggtacaagtgggaactacctgtgtacaaatggctctagtaaattactggactgctctgggtgtgaagccggcctttatcatcggacacagtcttggagactatgcagcccttaacacggccggtgttctatccaccagcgatacaatctatctttgtggccggcgtgctcagttgctgacgaaggaatgcaagattgggacacattcgatgctggccatcaaggcgtccctggcagaggtcaaacatttcctcagagacgagctccacgaagtctcttgtgttaacgcacctgcggagaccgtcgtcagcggccttgtcgctgatatcgacgagttggctcagaaatgctccacagagggtttgaagtcaaccaagctcaaggttccttacgcgttccattcctctcaggttgatcctatcttggaggccttcgaagatattgcccaaggtgtcaccttccacaagccgacaacacctttcgtctcagccctgttcggggaagtgatcaccgatgctaactgggagtgtctcggccccaagtacctgcgcgatcattgcagaaagacggtcaacttccttggcggcgtggaggctacgaggcatgcgaagctgaccaatgacaagactctgtgggttgagatcggctcacataccatttgctctggaatgatcaaagcaactcttggaccgcaagttacaacggttgcatctctacgccgcgaagaagatacctggaaggtcctttcgaacagtcttgcgagccttcatctggcgggtattgatatcaactggaagcaatatcaccaggactttagctcctctctccaggtcctccgcctcccagcctacaagtgggatctcaagaactactggattccctataccaacaacttctgcctgagcaagggcgctccagttgcgacagtagcggcagggccacagcatgagtacctgacaaccgcggctcagaaggtcattgagactcgaagtgatggagcaacagctacagtcgtgatagagaacgacattgctgatcccgagctcaaccgcgtcattcaaggccataaggtcaacggtactgctttgtgtccctcagtaagttaccgctcttgcccaacgactgcgttaagattcgtactaatcaggatatagtcactatatgccgacatctctcaaacgcttgcagagtatctcatcaaaaagtacaagcctgagtacgacggacttggactggatgtgtgtgaggtcacagtgccacgaccactgattgcgaaaggcggacagcagctctttagagtatctgcgacagcggattgggcggagaagaagacaacccttcagatatattcagtcactgcggaggggaagaagacggctgaccacgcaacttgcactgtccgattctttgactgcgctgctgcggaggcggaatggaaacgagtttcctaccttgtcaagaggagcattgaccgactgcatgatatcgccgaaaatggtgacgctcaccgtcttggtagaggcatggtttacaaactcttcgctgccttggttgattatgacgacaacttcaagtccattcgcgaggttattcttgacagtgaacagcacgaagcgactgcacgcgtcaagttccaagcaccacaaggcaatttccaccgaaacccgttctggattgacagttttggacacctgtctgggttcatcatgaacgcaagcgatgcaaccgactccaagaaccaggtctttgtcaatcacggatgggactccatgcgttgtttgaagaagttctcgcctgatgtcacctacaggacttatgttagaatgcagccttggaaagactccatctgggctggtgatgtctacgttttcgatggggatgatatcgttgcggtgtatggtgcagtcaaggtgagttcggcccgcgctcagttgcataagattcaaggtgctaatcattggtgtcacagttccaagccttatcacgcaagattctcgatacggtcctacctccagttggggcttcgaagggccccgccagaccagccgctagcgctcagaaggcggcccctgctgctgctgccagcaagagtcgtgctagcgccccggccccggcgaagcctgctgctaagcccagcgccccaagcttggtcaaacgggcacttaccatcctcgcagaggaagtgggtctgtctgaatccgagattacggatgatctggtcttcgcagactacggtgtggactcccttctttcgttgacggtcacgggcaggtatcgtgaagagctggatatcgatctcgaatcctccatcttcatcgaccagccgaccgtgaaagacttcaagcagttcttggccccaatgagccagggagaagccagcgatgggtccaccagtgacccagagtctagtagctccttcaatggtggctcttcaacagacgagtccagtgctgggtcccctgtcagctcaccaccaaatgagaaggttacgcaggtcgagcagcatgctacgataaaggagattcgcgccattttggccgatgagattggtgttacggaggaggagctgaaggacgatgagaacttgggagagatggggatggactctctgctttcgcttacggtgcttggtaggatccgtgagacattggatctggatctaccgggcgagttcttcatcgagaatcaaactctgaatgacgtggaggatgcattgggcctcaaacccaaggcagctcctgcgcctgcgcctgcgcctgctcccgtacccgcacccgtgtccgcgcccatattgaaggagcctgtccccaacgcaaactctaccatcatggcccgggcgagcccgcaccctcgatcaacctccattctgttgcaaggaaacccgaaaaccgcgaccaagaccctgttcctgttccctgatgggtctggctccgcaacatcgtatgcaaccattcccggagtgtccccggacgtgtgtgtctacggattgaactgcccgtacatgaagactccagagaagctcaagtatccccttgctgagatgacattcccctatctggccgagatccgccgcagacagcccaagggcccgtacaacttcggtggatggtctgcaggtggtatttgcgcctatgatgccgctcgctacctaatccttgaagagggcgaacaggttgaccgattgcttcttcttgactcgcccttccccattggcttagagaagttgcccactcggctgtacggcttcatcaactcaatgggtctctttggtgaaggcaacaaggctcccccggcctggttgctccctcatttcctggccttcattgattccctcgatacctacaaggccgtccccctcccctttgacgatccgaagtgggccaagaagatgccaaagacattcatggtctgggccaaggacggtatctgcagcaagccggatgacccgtggcccgagccggacccggacggcaagccggacacgagagagatggtctggctcctcaagaaccggaccgacatgggacccaacaagtgggacacactcgtcgggccccaaaacgtcggtggaatcactgtgatagagggtgcgaatcatttcaccatgactttgggacccaaggctaaagaattgggctcgttcattggcaacgccatggccaattaa

SEQ ID NO: 25 - DNA, 23 bp, Artificial Sequence
  Synthetic DNA
taacccaacccctcacttcacct

SEQ ID NO: 26 - DNA, 23 bp, Artificial Sequence
  Synthetic DNA
tggagacgtattccaggaaggct

SEQ ID NO: 27 - DNA, 22 bp, Artificial Sequence
  Synthetic DNA
tgtttggatctgatgccgaggc

SEQ ID NO: 28 - DNA, 20 bp, Artificial Sequence
  Synthetic DNA
ggcttgacgctgatcttggt

SEQ ID NO: 29 - DNA, 24 bp, Artificial Sequence
  Synthetic DNA
gaccaatgacaagactctgtgggt

SEQ ID NO: 30 - DNA, 22 bp, Artificial Sequence
  Synthetic DNA
tcttcttcccctccgcagtgac

SEQ ID NO: 31 - DNA, 24 bp, Artificial Sequence
  Synthetic DNA
tcaaatgcgcctatcacttcatgc

SEQ ID NO: 32 - DNA, 24 bp, Artificial Sequence
  Synthetic DNA
ccgccggttaatacgatgtcatat

SEQ ID NO: 33 - DNA, 21 bp, Artificial Sequence
  Synthetic DNA
gcagggcatcatcgacaaggt

SEQ ID NO: 34 - DNA, 23 bp, Artificial Sequence
  Synthetic DNA
ggtggtatcgagatcaggcaagg

SEQ ID NO: 35 - DNA, 19 bp, Artificial Sequence
  Synthetic DNA
tccatcatgacggctgcca

SEQ ID NO: 36 - DNA, 21 bp, Artificial Sequence
  Synthetic DNA
cgaactcggagctgatcttgc

SEQ ID NO: 37 - DNA, 21 bp, Artificial Sequence
  Synthetic DNA
gacgctgttctgtcgctttgt

SEQ ID NO: 38 - DNA, 23 bp, Artificial Sequence
  Synthetic DNA
gatcaggcagtatgggtggaagt

SEQ ID NO: 39 - DNA, 20 bp, Artificial Sequence
  Synthetic DNA
agtgggatctcaagaactac

SEQ ID NO: 40 - DNA, 20 bp, Artificial Sequence
  Synthetic DNA
ggcgggtgtcgatgtaccac

SEQ ID NO: 41 - DNA, 20 bp, Artificial Sequence
  Synthetic DNA
accatgccaatggattccgg

SEQ ID NO: 42 - DNA, 20 bp, Artificial Sequence
  Synthetic DNA
cgagcactggtagatgatga

SEQ ID NO: 43 - DNA, 26 bp, Artificial Sequence
  Synthetic DNA
gaattcccttgtatctctacacacag

SEQ ID NO: 44 - DNA, 32 bp, Artificial Sequence
  Synthetic DNA
tgaagaacgaataccgcgacatccaacccatc

SEQ ID NO: 45 - DNA, 20 bp, Artificial Sequence
  Synthetic DNA
tctcttcggcccttcatgag

SEQ ID NO: 46 - DNA, 20 bp, Artificial Sequence
  Synthetic DNA
cggtgtcaaagtccagatgg

SEQ ID NO: 47 - DNA, 20 bp, Artificial Sequence
  Synthetic DNA
ctgccccaggatactgctga

SEQ ID NO: 48 - DNA, 20 bp, Artificial Sequence
  Synthetic DNA
ctgccccaggatactgctga

SEQ ID NO: 49 - DNA, 20 bp, Artificial Sequence
  Synthetic DNA
tctcttcggcccttcatgag

SEQ ID NO: 50 - DNA, 957 bp, Artificial Sequence
  Synthetic DNA
atgtccaccccatccgaccccaaacccaccctagtccggtggtacatcgacacccgccccctaaccgccacctccaacgccctccccctcctcgacaccctccaacccaccgaacaacaagccatccagaaatactatcacctgaaggacagacacatgtccctagcctccaacctcctcaaatacctcttcatccaccgcacctgccgcatcccctggtcccaaatcaccatcagccggaccccagccccgcaccatcgaccctgcttcatcccatccgcagcagtcctctccgcctccagctcctcaatcccaacggtcgaattcaacgtcagccaccaggactcaatggtcgccttagccggcactaccatcccacccaatgccaaaaaagaagaaatccaagtaggaatcgacataaccagcacaacggaacacctccgctccccgcgcaacccgtcccctcccacccggtcagccttcctggaatacgtctccatattcacggagatattctcggcgagggaactatctttgattaaatcgctcacgcatcctgtgtatcattatccggtgtcgtcgacggagggggtggtgtatagttatcgggtgttttttgcgtattgggcgcttaaggaggcgtatattaagatgacgggggaagcgttgttagcggagtggttgagggagttggagtttttggatgtgagggttccggataaggatggggaggtgtataggggggtcagggtggagatgaaggggagggaggtcggggatgttagggttgaggtgcaggcgtttgagggggggtatttggttgctacggcggccagagggggaggtgttgggaggatagagggagaagggaaagatgtgtgggagggattgaggaggttggatgttgaaggggatgttagggtttgtgcggaggggaggtgtgcttgtttggagtaa

SEQ ID NO: 51 - DNA, 957 bp, Artificial Sequence; identical to SEQ ID NO: 50
  Synthetic DNA

SEQ ID NO: 52 - DNA, 2044 bp, Artificial Sequence
  Synthetic DNA
atgatggtcgcgtggtggtctctatttctgtacggccttcaggtcgcggcacctgctttggctgcaacgcctgcggactggcgatcgcaatccatttatttccttctcacggatcgatttgcaaggacggatgggtcgacgactgcgacttgtaatactgcggatcaggtgtgttgttacctactagctttcagaaagaggaatgtaaactgacttgatatagaaatactgtggtggaacatggcagggcatcatcgacaaggtaaattgcccctttatcaaaaaaaaagaaggaaaagcagaagaaaaataaaataaaaagaactctagtcctaaccatcacatagttggactatatccagggaatgggcttcacagccatctggatcacccccgttacagcccagctgccccagaccaccgcatatggagatgcctaccatggctactggcagcaggatatgtaagtcgatttctttaaatatctacctgtcatcttttacatcaatatgaactaacttgatggttttagatactctctgaacgaaaactacggcactgcagatgacttgaaggcgctctcttcggcccttcatgagagggggatgtatcttatggtcgatgtggttgctaaccatatggttcgtggtcctttgcaactgacttcgcggatatggttcatttcagtactgacaatgagtaatatcagggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccagtaagatatttctccctcattctacaacttggctgatcgatgatacttacgaaatcagttgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatgtatggttcctccaaccatgagccttcttgcaagtctcatctcctaacgaaacggctaaaaccagttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcgtaagtcttcccttttattttccgttcccaatttccacacagaaccccacctaacaagagcaaagttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaaggtaagcacaacctctaagcataccctaatggcctatcttcagagtatctgacacaagagactaatcactggcaatacagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgtga

SEQ ID NO: 53 - DNA, 1975 bp, Artificial Sequence
  Synthetic DNA
atgagattatcgacttcgagtctcttcctttccgtgtctctgctggggaagctggccctcgggctgtcggctgcagaatggcgcactcagtcgatttacttcctattgacggatcggttcggtaggacggacaattcgacgacagctacatgcgatacgggtgaccaagtacgttggtattgcaggacttccatcattcatctactgacttgaatagatctattgtggtggcagttggcaaggaatcatcaaccatgtttgtgatcacttcatactatccgctgtgcgcgtgtctgactttatttgctgcagctggattatatccagggcatgggattcacggccatctggatctcgcctatcactgaacagctgccccaggatactgctgatggtgaagcttaccatggatattggcagcagaagatgtatgcgctcctccttcccatatcgtaggcttactctcaggcggcgactgacttgacagatacgacgtgaactccaacttcggcactgcagatgacctcaagtccctctcagatgcgcttcatgcccgcggaatgtacctcatggtggacgtcgtccctaaccacatggtaagtgctgcttcagcatccttatcagtgaactccaagtgccaacgctaactgtaccagggctacgccggcaacggcaacgatgtagactacagcgtcttcgaccccttcgattcctcctcctacttccacccatactgcctgatcacagattgggacaacttgaccatggtccaagattgttgggagggtgacaccatcgtatctctgccagacctaaacaccaccgaaactgccgtgagaacaatctggtatgactgggtagccgacctggtatccaattattcaggtgcgaattccaacccaatttaaaataaccatatactaagtgaaatcaccagtcgacggactccgcatcgacagtgtcctcgaagtcgaaccagacttcttcccgggctaccaggaagcagcaggtgtctactgcgtcggcgaagtcgacaacggcaaccctgccctcgactgcccataccagaaggtcctggacggcgtcctcaactatccgatgtacatccccctatacattgttcattagatcttcgctaactccaaccagctactggcaactcctctacgccttcgaatcctccagcggcagcatcagcaacctctacaacatgatcaaatccgtcgcaagcgactgctccgatccgacactactcggcaacttcatcgaaaaccacgacaatccccgtttcgcctcgtatgtcccaccccctcccctccctacaatcacactcactaatacatctaacagctacacctccgactactcgcaagccaaaaacgtcctcagctacatcttcctctccgacggcatccccatcgtctacgccggcgaagaacagcactactccggcggcaaggtgccctacaaccgcgaagcgacctggctttcaggctacgacacctccgcagagctgtacacctggatagccaccacgaacgcgatccgcaaactagccatctcagctgactcggcctacattacctacgcggttcgtccttccctcccaccctttaccccccaccctacaaacatcccacatactaacaacatttcaataatgaaatagaatgatgcattctacactgacagcaacaccatcgcaatgcgcaaaggcacctcagggagccaagtcatcaccgtcctctccaacaaaggctcctcaggaagcagctacaccctgaccctcagcggaagcggctacacatccggcacgaagctgatcgaagcgtacacatgcacatccgtgaccgtggactcgagcggcgatattcccgtgccgatggcgtcgggattaccgagagttcttctgcccgcgtccgtcgtcgatagctcttcgctctgtggcgggagcggaagattatacgtcgagtaa

SEQ ID NO: 54 - DNA, 1539 bp, Artificial Sequence
  Synthetic DNA
atggtcgtcttcagcaaaaccgctgccctcgttctgggtctgtcctccgccgtctctgcggcgccggctcctactcgcaagggcttcaccatcaaccagattgcccggcctgccaacaagacccgcaccatcaacctgccaggcatgtacgcccgttccctggccaagtttggcggtacggtgccccagagcgtgaaggaggctgccagcaagggtagtgccgtgaccacgccccagaacaatgacgaggagtacctgactcccgtcactgtcggaaagtccaccctccatctggactttgacaccggatctgcagatctgtaagcttccctgctcgggtgttcgggcaaatcgtgactaacctggactagctgggtcttctcggacgagctcccttcctcggagcagaccggtcacgatctgtacacgcctagctccagcgcgaccaagctgagcggctacacttgggacatctcctacggtgacggcagctcggccagcggagacgtgtaccgggatactgtcactgtcggcggtgtcaccaccaacaagcaggctgttgaagcagccagcaagatcagctccgagttcgttcagaacacggccaatgacggccttttgggactggcctttagctccatcaacactggtgagtcaatcctacatcagccgggttgacctacctgctgaccgatacacagtccagcccaaggcgcagaccaccttcttcgacaccgtcaagtcccagctggactctccccttttcgccgtgcagctgaagcacgacgcccccggtgtttacgactttggctacatcgatgactccaagtacaccggttctatcacctacacggatgccgatagctcccagggttactggggcttcagcaccgacggctacagtatcggtgacggcagctccagctccagcggcttcagcgccattgctggtaagaaccgccttcatttaacacacaacttgtccacctctttactaactagtgtatagacaccggtaccaccctcatcctcctcgatgacgaaatcgtctccgcctactacgagcaggtttctggcgctcaggagagcgaggaagccggtggctacgttttctcttgctcgaccaacccccctgacttcactgtcgtgattggcgactacaaggccgttgttccgggcaagtacatcaactacgctcccatctcgactggcagctccacctgctttggcggtatccagagcaacagcggtctgggactgtccatcctgggtgatgttttcttgaagagccagtacgtggtcttcaactctgagggccctaagctgggattcgccgctcaggcttagattatccactgaagtggagtctatgatctgctgattgatccctcgacgatgaactacatgtggaaatgcatagcagacgagggtgatggtgatgatgttgatttgatgatgacccgtacatacttgatgaagctcggtacatatgcaaatgtgactgtatctatgtgatgaatatatgtatccatctcatgg

SEQ ID NO: 55 - DNA, 2522 bp, Artificial Sequence
  Synthetic DNA
atggcggacggcaacccacatcgggaagatgaggcggccgaggaagaagaggagattgatgagactgtacgcaaatttacccatgaacttggactggaactctggaactgacaataagatcagagctacaaaccagtcaaagatgcggtcctcttcgcaatcgatgtcagcgattccatgttgacgcctcgcccctcagcagatcctaagaaacacacccaagaatcacccaccacggcagcgctcaaatgcgcctatcacttcatgcaacaacgaatcatatcaaatccacaagacatgatgggtgttttgctgttcgggacccaggcgtccaagttctttgaagaagatgaagacagtcggggagacctgtcctaccccaactgctacctcttcactgatctggatgttccttcggctcatgaggtcaaaggacttcgagcactggtagatgatgaaggagactcaagggaggttctatctccagcgaaagagcaggtctctatggcaaacgtcctattttgcgccaaccagatattcacatccagagcgccaaatttcctctcccggcgtttgttcatcataaccgacaatgacaacccccatggtgatgataaaaccctgcggtcagcggcgactgtacgtgctaaggatctttacgatcttggtgtcacaattgagctgtttccgatctcacgccctgagcatgagttcaagaacagcaagttctatgacgtaagctatcatactctatagcaaagtggcaggggtcgatactcactacagatacaaaggatattatctacaagtcattgcccagcgatccagaggcgcctgcatatctacaatctgattcaaaagcggcgactgcgaccggggacgggatttcactcctcaacacgcttctgtccagtattaattcgagaacggttccgcgtcgcactcatttttcgaacatgcctttagaacttggcccagacttcagaatttcggtatcgggctatatactcttacgaaggcaagcgcccgctagaaactccttcatctggctgaacggcgagaagcctgtggtcgcgaaaggagtgacttcccactccgcagatgatactggccggactgtcgagaaatgggagatcagaaaggcatataagttcggtggcgaccaagtaaccttttcgcctgatgagcagaaggcgcttagggatttcggtgagccagtaatccgggttattgggttcaagcctatcactgcgcttccattctgggcaaacgtcaagcacccatattttatctatccatccgaggaagactatgtaggctcctcgcgagtattttccgcattgcatcagactcttttgcgttccaagaagatggcactcgtctggttcattgcacgcaagggtgctggccccgttctcgccgctatgatcgcaggcgaagaaaagcttgatgagaatggcgtacaaaaataccctcctggcatgtggattcttcccctccccttcgcagacgatatccggcagaaccccgaaacaacgttgaatgtcgccccggagtcattgattgatcagatgcgcgtgatcgtccagcaactgcagctgccgaagggagtgtacgagcctctcaaataccccaatccatgtaagtcacttctgtcttgcattgctcgtatacgatgaacgagaagctgacagcccgtgatcagcccttcaatggcattaccgcatcctacaagctctcgcattagacgaagatctccccgaaaaaccagaagacaaaaccattccgaaataccgccaaatcgacaaggtaaatccaccacacccaacacgagaaataaccctccaggcgtccaacttactgacaattgcaccacagcgcgccggtgactacgtattatcctgggccgacgaactcgaaaagcaatacgccaaaacctcagcagcggcccctcgcccaaccagcaccctcgtgaaacgaggatcaaaagaccgagcaagcgaaaccgaggactccaagccatcgaaaaagatcaaggttgaggaagactctggaagcctagaggaggaagtccgcaggcatcacaagaagggaacgctatccaaggtaagccaccacaggctttctacacgtcctcgtgatggcaaatatgacatcgtattaaccggcggttttctagcttacggtcgctatcctcaaggacttcttgacttccaatggacgctcaaatgccggtaagaaggcggatcttattgagcgggtagaggagttcttggagcagtgacatggcgggattgttggattcgctagtgcgcttctgttggtggatgtcgttatgtggtgtcttatctcgggttaggcgttcgtgacctgaggacatgagcttgtaattaatgatgggttggatgtcgcggtattcgttcttcagcgaaacgtaatggacacgtattttaggcgatgtacagttataaaaaatcgaattcgctgggctagccggacatgtcaaaacgaagagcattagg

SEQ ID NO: 56 - DNA, 21 bp, Artificial Sequence
  Synthetic DNA
cccaagaagaagcgcaaggtc